(19) United States
(12) Patent Application Publication
(10) Pub. No.: US 2016/0113546 A1
(43) Pub. Date: Apr. 28, 2016

(54) METHODS AND SYSTEMS FOR PROCESSING MRI IMAGES TO DETECT CANCER

(71) Applicant: Khalifa University of Science, Technology & Research, Abu Dhabi (AE)

(72) Inventors: Ehab Najeh Salahat, Abu Dhabi (AE); Hani Hasan Mustafa Saleh, Abu Dhabi (AE); Safa Najeh Salahat, Abu Dhabi (AE); Andrzej Stefan Sluzek, Abu Dhabi (AE); Mahmoud Al-Qutayri, Abu Dhabi (AE); Baker Mohammad, Abu Dhabi (AE); Mohammed Ismail Elnaggar, Abu Dhabi (AE)

(21) Appl. No.: 14/521,954

(22) Filed: Oct. 23, 2014

(51) Int. Cl. A61B 5/055; A61B 5/00; G06T 7/00

(52) U.S. Cl. CPC A61B 5/055; G06T 7/0014; A61B 5/7282; G06T 2207/10088; G06T 2207/30096

(57) ABSTRACT

Methods and systems process an MRI image to detect cancer. A method includes forming a series of binary threshold intensity images from an MRI image of a patient. Each of the binary threshold intensity images is based on a respective intensity. The binary threshold intensity images are processed to identify one or more bright extremal regions in which image pixels have the same value, and for which corresponding image pixels in the MRI image have a higher intensity than surrounding image pixels in the MRI image. One or more bright maximally stable extremal regions are selected from the identified bright extremal regions based on change in area of one or more respective bright extremal regions for different binary threshold images in the series. At least one of the selected one or more bright maximally stable extremal regions may be identified as potentially cancerous.

### BACKGROUND

It is estimated that 14.1 million new cases of cancer occurred globally in 2012. It is also estimated that cancer caused about 8.2 million deaths or 14.6% of all human deaths in 2012. Common types of cancer include lung cancer, prostate cancer, colorectal cancer, stomach cancer, breast cancer, cervical cancer, skin cancer, acute lymphoblastic leukemia, brain tumors, and non-Hodgkin lymphoma. The financial cost of cancer has been estimated to be $1.16 trillion US dollars per year as of 2010

Cancer detection is typically a very sensitive, complicated, and time consuming task. Recently, lots of research is tackling the task of detecting diseases through processing digitized medical images from diversified sources such as X-ray, Ultra-sound, magnetic resonance imaging (MRI), etc. For example, some research has been directed to detection of cancer using MRI images. The research is primarily focusing on detection of one type of cancer (e.g., brain, breast, etc.) rather than detecting multiple types of cancer using a single technique. More importantly, the detection of the cancerous parts of the MRI image in this research is based on techniques that are computationally intensive, hardware demanding, and time consuming as the techniques require many pre-operative and post-operative complicated processes before a final cancer detection is made. For example, in D. Kwon, M. Niethammer, H. Akbari, M. Bilello, C. Davatzikos and K. M. Pohl, “PORTR: Pre-Operative and Post-Recurrence Brain Tumor Registration,” IEEE Trans. on Medical Imaging, vol. 33, no. 3, pp. 651-667, March, 2013, the required time for the processing for tumor detection is 3.5 hours and the referenced technique is only targeting brain cancer. Other techniques, such as (1) M. B. Cuadra, C. Pollo, A. Bardera, O. Cuisenaire, J.-G. Villemure and J.-P. Thiran, “Atlas-Based Segmentation of Pathological MR Brain Images Using a Model of Lesion Growth,” IEEE Trans. on Medical Imaging, vol. 23, no. 10, pp. 1301-1314, October, 2004; and (2) J. J. Corso, E. Sharon, S. Dube, S. El-Saden, U. Sinha and A. Yuille, “Efficient Multilevel Brain Tumor Segmentation With Integrated Bayesian Model Classification,” IEEE Trans. on Medical Imaging, vol. 27, no. 5, pp. 629-640, May, 2008, require additional classifiers and neural networks analysis for cancer detection, which is also computationally intensive and time consuming.

In view of the huge impact of cancer and the time and expense associated with existing techniques for detecting cancer, improved cancer detection approaches remain of interest. The approaches and related systems for detecting cancer presented herein address many of the shortcomings of existing cancer detection techniques.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

The approaches and systems described herein process one or more magnetic resonance imaging (MRI) images to detect cancer. In many embodiments, a maximally stable extremal region (MSER) algorithm is employed to detect bright MSERs in which the pixels are brighter than surrounding pixels in the MRI image. The intensity of each bright MSER region detected can be compared to minimum and maximum intensity values to ensure that the bright MSER region detected corresponds to an intensity range indicative of a potentially cancerous region. The approaches and systems described herein provide for fast, efficient, and potentially highly accurate detection of many different types of cancer.

Thus, in one approach, a method is described for processing a magnetic resonance imaging (MRI) image to detect cancer in a patient. The method includes receiving an MRI image of the patient. A series of binary threshold intensity images are formed from the MRI image. Each of the series of binary threshold intensity images is based on a respective intensity in a series of intensities. The series of binary threshold intensity images is processed to identify one or more bright extremal regions in which image pixels in the respective binary threshold intensity image have the same value, and for which corresponding image pixels in the MRI image have a higher intensity than surrounding image pixels in the MRI image. One or more bright maximally stable extremal regions are selected from the identified bright extremal regions based on change in area of one or more respective bright extremal regions for different binary threshold images in the series. At least one of the selected one or more bright maximally stable extremal regions may be identified as potentially cancerous.

In many embodiments of the method, parameters are employed to ignore identifying detected regions that are not likely to be cancerous. For example, in many embodiments: a) no bright maximally stable extremal regions having a corresponding image intensities in the MRI image less than a minimum intensity value and/or greater than a maximum intensity value are identified as potentially cancerous; and b) no bright maximally stable extremal regions having an area less than a minimum area and/or greater than a maximum area are identified as potentially cancerous.

All bright MSERs identified can be restricted to those having area with a specified size stability between different images in the series of binary threshold images. For example, in many embodiments, no bright extremal regions having a change in area of greater than a maximum area change tolerance for the different images in the series of binary threshold intensity images are selected as the bright maximally stable extremal regions.

In many embodiments of the method, parameters are generated that are descriptive of the location and size of the at least one potentially cancerous region. For example, the parameters generated can define an ellipse approximating the respective potentially cancerous region.

In many embodiments of the method, expansion or contraction of a cancerous region is tracked via processing of a sequence of two or more MRI images taken at different times. For example, the method can further include: a) receiving a second MRI image of the patient; b) forming a second series of binary threshold intensity images from the second MRI image, each of the second series of binary threshold intensity images being based on a respective intensity in a second series of intensities; c) processing the second series of binary threshold intensity images to identify one or more second image bright extremal regions in which image pixels in the respective binary threshold intensity image have the same value, and for which corresponding image pixels in the second MRI image have a higher intensity than surrounding image pixels in the second MRI image; d) selecting one or more second image bright maximally stable extremal regions from the identified second image bright extremal regions based on change in area of one or more respective second image bright extremal regions for different binary threshold images in the second series; and d) identifying at least one of the selected one or more second image bright maximally stable extremal regions as corresponding to at least one of the one or more bright maximally stable extremal regions identified as potentially cancerous. In many embodiments, the method further includes determining a change in area of a region identified as potential cancerous based on the MRI image and the second MRI image.

The method is suitable for the detection of many different types of cancers and/or tumors. For example, in many embodiments, the one or more bright maximally stable extremal regions identified as potentially cancerous are identified as being potentially one of the group of cancers consisting of: a) bladder cancer, b) breast cancer, c) colon cancer, d) rectal cancer, e) endometrial cancer, f) kidney cancer, g) leukemia, h) lung cancer, i) melanoma cancer, j) non-Hodgkin lymphoma cancer, k) pancreatic cancer, l) prostate cancer, m) thyroid cancer, and n) brain cancer.

In another aspect, a system is described for processing a magnetic resonance imaging (MRI) image to detect cancer in a patient. The system includes one or more processors and a tangible memory storage device. The memory storage device stores instructions that when executed by the one or more processors cause the system to: a) receive an MRI image of the patient; b) form a series of binary threshold intensity images from the MRI image, each of the series of binary threshold intensity images being based on a respective intensity in a series of intensities; c) process the series of binary threshold intensity images to identify one or more bright extremal regions in which image pixels in the respective binary threshold intensity image have the same value, and for which corresponding image pixels in the MRI image have a higher intensity than surrounding image pixels in the MRI image; d) select one or more bright maximally stable extremal regions from the identified bright extremal regions based on change in area of one or more respective bright extremal regions for different binary threshold images in the series; and e) identify at least one of the selected one or more bright maximally stable extremal regions as potentially cancerous.

In many embodiments of the system, parameters are employed to ignore identifying detected regions that are not likely to be cancerous. For example, in many embodiments: a) no bright maximally stable extremal regions having a corresponding image intensities in the MRI image less than a minimum intensity value and/or greater than a maximum intensity value are identified as potentially cancerous; and b) no bright maximally stable extremal regions having an area less than a minimum area and/or greater than a maximum area are identified as potentially cancerous.

All bright MSERs identified can be restricted to those having area with a specified size stability between different images in the series of binary threshold images. For example, in many embodiments of the system, no bright extremal regions having a change in area of greater than a maximum area change tolerance for the different images in the series of binary threshold intensity images are selected as the bright maximally stable extremal regions.

In many embodiments of the system, parameters are generated that are descriptive of the location and size of the at least one potentially cancerous region. For example, the parameters generated can define an ellipse approximating the respective potentially cancerous region.

In many embodiments of the system, expansion or contraction of a cancerous region is tracked via processing of a sequence of two or more MRI images taken at different times. For example, in many embodiments of the system, the instructions, when executed by the one or more processors, further cause the system to: a) receive a second MRI image of the patient; b) form a second series of binary threshold intensity images from the second MRI image, each of the second series of binary threshold intensity images being based on a respective intensity in a second series of intensities; c) process the second series of binary threshold intensity images to identify one or more second image bright extremal regions in which image pixels in the respective binary threshold intensity image have the same value, and for which corresponding image pixels in the second MRI image have a higher intensity than surrounding image pixels in the second MRI image; d) select one or more second image bright maximally stable extremal regions from the identified second image bright extremal regions based on change in area of one or more respective second image bright extremal regions for different binary threshold images in the second series; and e) identify at least one of the selected one or more second image bright maximally stable extremal regions as corresponding to at least one of the one or more bright maximally stable extremal regions identified as potentially cancerous. In many embodiments of the system, the instructions, when executed by the one or more processors, further cause the system to determine a change in area of a region identified as potential cancerous based on the MRI image and the second MRI image.

The system is suitable for the detection of many different types of cancers and/or tumors. For example, in many embodiments of the system, the one or more bright maximally stable extremal regions identified as potentially cancerous are identified as being potentially one of the group of cancers consisting of: a) bladder cancer, b) breast cancer, c) colon cancer, d) rectal cancer, e) endometrial cancer, f) kidney cancer, g) leukemia, h) lung cancer, i) melanoma cancer, j) non-Hodgkin lymphoma cancer, k) pancreatic cancer, l) prostate cancer, m) thyroid cancer, and n) brain cancer.

For a fuller understanding of the nature and advantages of the present invention, reference should be made to the ensuing detailed description and accompanying drawings.

### BRIEF DESCRIPTION OF THE DRAWINGS

Various embodiments in accordance with the present disclosure will be described with reference to the drawings, in which:

FIG. 1A is a simplified schematic diagram of acts of a method of processing a magnetic resonance imaging (MRI) image to detect cancer, in accordance with many embodiments.

FIG. 1B is a simplified schematic diagram of acts of a method of processing MRI images to monitor cancer growth/shrinkage, in accordance with many embodiments.

FIG. 2 and FIG. 3 show example detection results for brain cancer, in accordance with many embodiments.

FIG. 4 and FIG. 5 show example detection results for breast cancer, in accordance with many embodiments.

FIG. 6 shows an example detection result for brain cancer that took about 13 seconds of processing time to generate, in accordance with many embodiments.

FIG. 7 shows an example detection result for brain cancer that took about 15 seconds of processing time to generate, in accordance with many embodiments.

FIG. 8 shows an example detection result for breast cancer that took about 6 seconds of processing time to generate, in accordance with many embodiments.

FIG. 9 shows an example detection result for breast cancer that took about 5.2 seconds of processing time to generate, in accordance with many embodiments.

FIG. 10 is a simplified schematic diagram illustrating an approach for processing an MRI image to detect cancer, in accordance with many embodiments.

FIG. 11 is a schematic diagram illustrating an approach for processing a binary-threshold image to detect contiguous regions, in accordance with many embodiments.

FIG. 12 is a schematic diagram illustrating an approach for detecting maximally stable extremal regions in an MRI image, in accordance with many embodiments.

FIG. 13 illustrates a scanning approach for use in determining region sizes for identified extremal regions, in accordance with many embodiments.

FIG. 14 illustrates elliptical approximation of a maximally stable extremal region, in accordance with many embodiments.

FIG. 15 is a simplified schematic diagram of a system for processing a magnetic resonance imaging (MRI) image to detect cancer, in accordance with many embodiments.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

The approaches and systems described herein are directed to processing medical images to detect cancer. In many embodiments, an MRI image is processed to detect one or more bright maximally stable extremal regions (MSERs) that are potentially cancerous. The approaches and systems described herein are suitable for the detection of many types of cancer via a single approach. For example, cancers and tumors detectable using the approaches and systems described herein include, but are not limited to, brain cancer, bladder cancer, breast cancer, colon cancer, rectal cancer, endometrial cancer, kidney cancer, leukemia cancer, lung cancer, melanoma cancer, non-Hodgkin lymphoma cancer, pancreatic cancer detection, prostate cancer, and thyroid cancer.

The approaches and systems described herein are computationally and time efficient. In many embodiments, no pre-processing or post-processing of the MRI image(s) is employed. Most importantly, the approaches and systems described herein may provide a very accurate technique for cancer and tumor detection through accurate and efficient detection of a cancerous region(s) in a target area. Additionally, the approaches and systems described herein are easily implemented and may reduce the amount of detection related processing significantly.

Moreover, the approaches and systems described herein resolves many of the issues that existing cancer detections approaches suffer from. For example, in many embodiments, the approaches and system described herein: a) employ an easily configurable Maximally Stable Extremal Regions image detector algorithm; b) can detect cancerous regions at early cancer growth stages or any stage; c) can monitor the cancerous regions progression/diminishing rate using a sequence of MRI images taken at different times; d) provide a highly accurate detection of a cancerous region(s); e) can be utilized for the detection of multiple cancer types, for example, brain cancer, breast cancer, etc.; f) perform the detection task in a very fast way in a matter of seconds instead of hours; g) do not require pre-operative or post-operative processing; h) can be implemented fairly easily; i) simplify image processing as only the bright or dark intensity images are needed and hence the MSER detector need only to be run once based on either one of them; j) are very power efficient by avoiding pre- or post-operative processing and by eliminating the need to run the MSER algorithm twice on the two different version of the intensity image; and k) are memory efficient by detecting the minimal number of MSERs that correspond to the cancerous regions (typically less than 10 regions).

In many embodiments, the MSER algorithm employed is an image region detection algorithm. Operational parameters of the MSER algorithm can be selected to control its detection capability, namely the maximum and minimum size (number of pixels) in the detected regions, the threshold intensity increment value, and the maximum region area variation. Different configuration of these parameters may produce different detected regions. In many embodiments, the MSER algorithm employed takes an intensity image and processes it to detect what are referred to herein as “bright MSERs.” In standard MSER algorithms, the intensity image is then inverted and the inverted intensity image is processed to detect dark MSERs.

In many embodiments, the MSER algorithm employed is configured for the detection of bright MSER(s). Because cancerous regions typically appear as bright regions in MRI images, detection of bright MSERs (regions that have brighter pixels than the surrounding pixels) is used to identify potentially cancerous regions. By detecting only the bright MSERs, the MSER algorithm employed in many embodiments does not detect the dark MSERs, thereby cutting processing by nearly 50%.

In many embodiments, an additional detection parameter is used in the MSER algorithm employed to ignore extra bright MSERs, which typically do not correspond to potentially cancerous regions. The additional detection parameter can be implemented as a range of intensity values for bright MSERs that correspond to regions that are potentially cancerous. For example, once a bright MSER is identified, the intensity values (from the MRI image) are compared to a maximum intensity parameter value and a minimum intensity parameter value to determine if the bright MSER identified should be identified as potentially cancerous and stored. If the intensity values are not within the range of interest, the identified bright MSER can be ignored.

In many embodiments, the MSER parameters employed makes the MRI algorithm employed suitable to detect cancerous regions at their different stages. Furthermore, with a sequence of MRI frames for that patient, the expansion or contraction of the identified cancerous region can be easily monitored.

Bright MSER Detection

The detection of one or more bright maximally stable extremal regions (MSERs) is described as follows. Starting with an M×N empty grid that corresponds to an M×N intensity image, all entries of the empty grid are initially set to the same first binary value (e.g., a value representing the color black). The resulting starting M×N grid (with all entries set to the first binary value) serves as a first of a series of what are referred to herein as binary threshold intensity images. The remaining of the series of binary threshold intensity images are generated by progressively varying an intensity threshold by a threshold increment delta (Δ) from the maximum intensity to the minimum intensity used, for example, from 255 to 0 in steps equal to the threshold increment delta (Δ). At each threshold, all pixels in the corresponding binary threshold intensity image corresponding to pixels in the intensity image with values that are above the current threshold are assigned a second binary value (e.g., a value representing the color white) and the remaining pixels in the corresponding binary threshold intensity image are assigned the first binary value (e.g., a value representing the color black). As the threshold decreases from the maximum intensity value to the minimum intensity value, some white regions will appear, some of the white regions will merge, and ultimately all of the white regions will merge to produce a totally white image at least when the threshold reaches the minimum intensity value. During this process, the size of each white region (i.e., its cardinality Q(t)) is monitored as a function of threshold value t. A bright MSER is detected if q(t) defined in equation (1) below has a local minimum.

q(t)=[Q(t+Δ)/Q(t−Δ)]/Q(t) Equation (1)

The detected bright MSERs in this case correspond to the white regions. The word ‘extremal’ refers to the property that all pixels inside the bright MSER have higher intensity than all the pixels on its outer boundary.

The bright MSER detection is controlled by four main parameters, namely the threshold increment Δ, the minimum and maximum size of each region, and the maximum area variation defined by the stability function q(t). The lower the value of Δ, the algorithm is slower but produces more accurate results. Typically, Δ is selected in the range of 4 to 7 wherein the possible intensity values vary from 255 to 0.

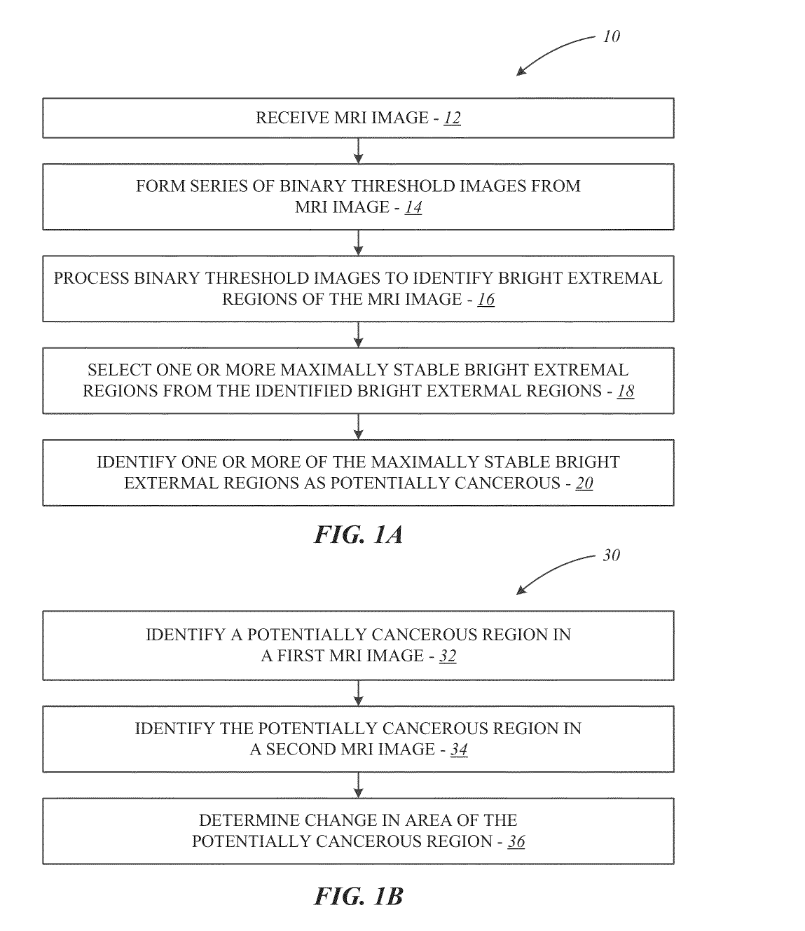

Referring now to the drawings, in which like reference numerals represent like parts throughout the several views, FIG. 1A shows a method 10 of processing an MRI image to detect cancer in a patient, in accordance with many embodiments. The method 10 includes receipt of an MRI image of the patient (act 12). A series of binary threshold images is formed from the MRI image (act 14). In any embodiments, the series of binary threshold images are formed using a series of intensities that progresses from a maximum intensity to a minimum intensity. As a result, regions that appear and grow in the series of threshold images correspond to regions in the MRI image in which the pixels have a higher intensity than surrounding pixels in the MRI image. The binary threshold images are processed to identify bright extremal regions in the MRI image (act 16). One or more bright MSERs are selected from the identified bright extremal regions (act 18). One or more of the bright MSERs are then identified as being potentially cancerous (act 20). The intensity of each of the bright MSERs can be checked to determine if the intensity is within a range indicative of the bright MSER being potentially cancerous. For example, the intensity of each of the bright MSERs can be checked relative to a minimum intensity value and a maximum intensity value to determine if the intensity is within a range of intensities indicative of the region being potentially cancerous.

FIG. 1B shows a method 30 for processing MRI images to monitor the size and/or location of a regions identified as being potentially cancerous. The method 30 includes identifying a potentially cancerous region(s) in a first MRI image (act 32). For example, the method 10 described herein can be used to identify the potentially cancerous region(s) in the first MRI image. The method 30 further includes identifying the potentially cancerous region in a second MRI image corresponding to the potentially cancerous region identified in the first MRI image. The method 10 can be used to identify the potentially cancerous region in the second MRI image. The size and/or location of the potentially cancerous region in the second MRI image is compared to the size and/or location of the potentially cancerous region in the first MRI image to determine a change in area of the region (act 36).

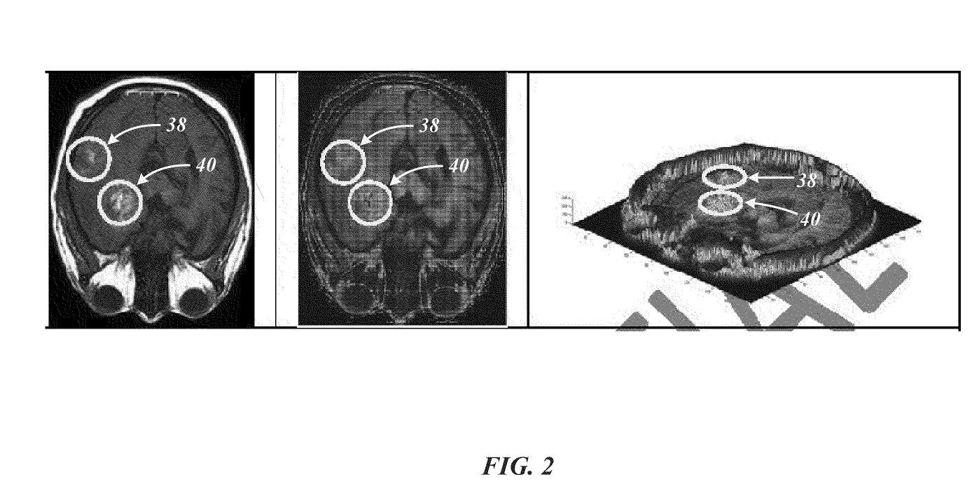

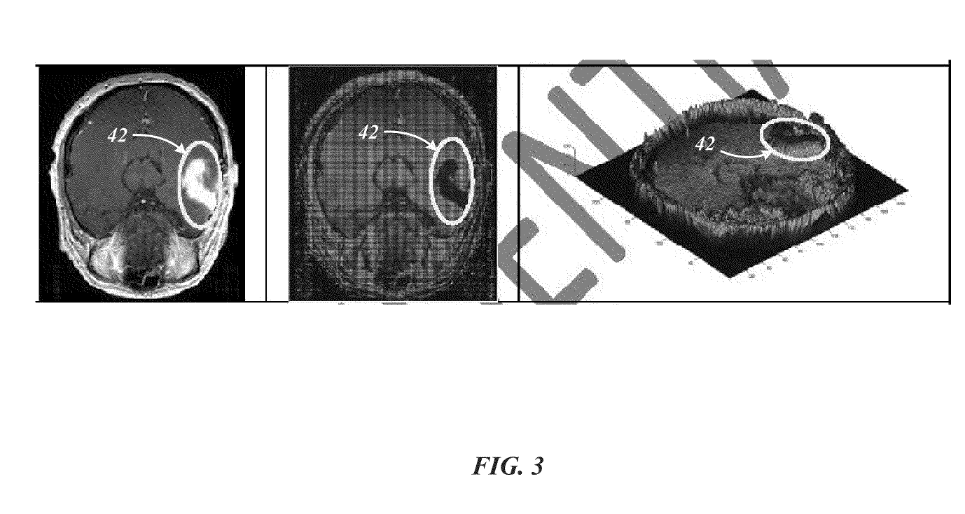

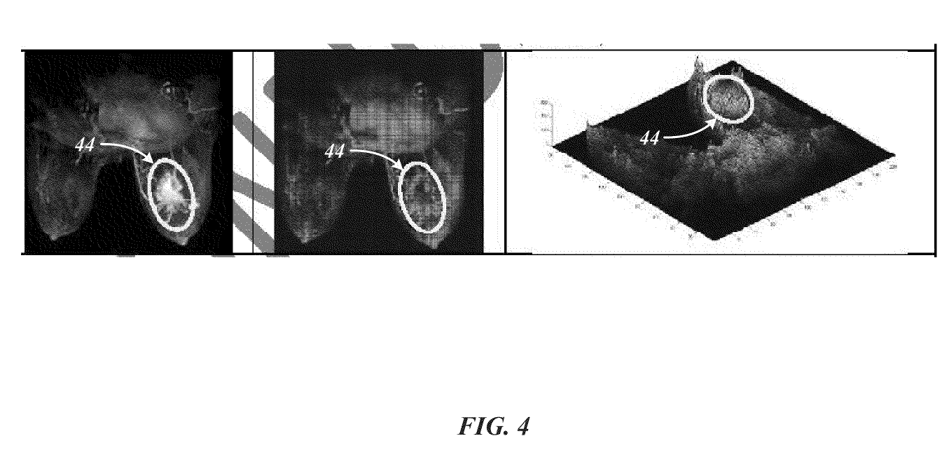

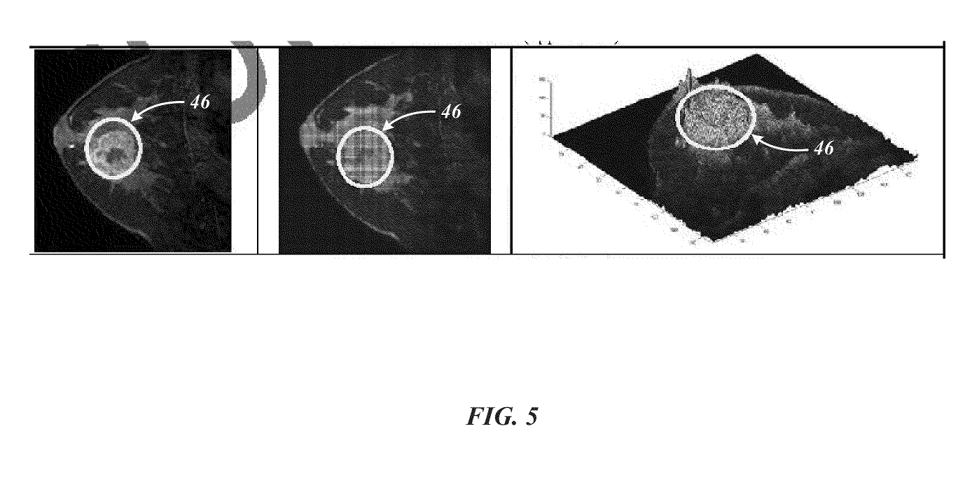

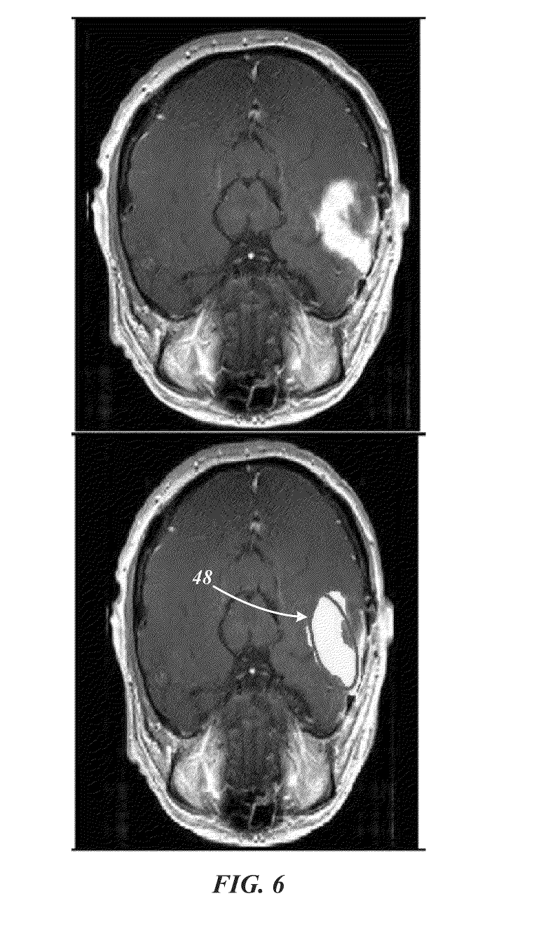

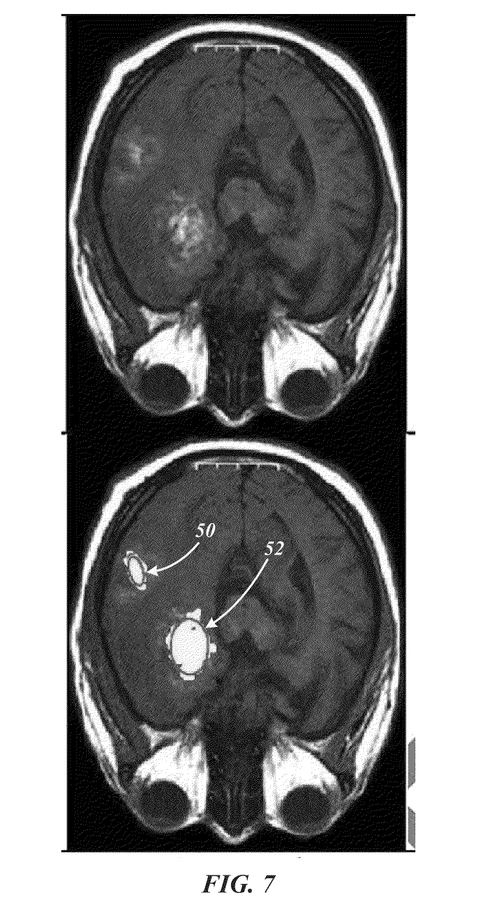

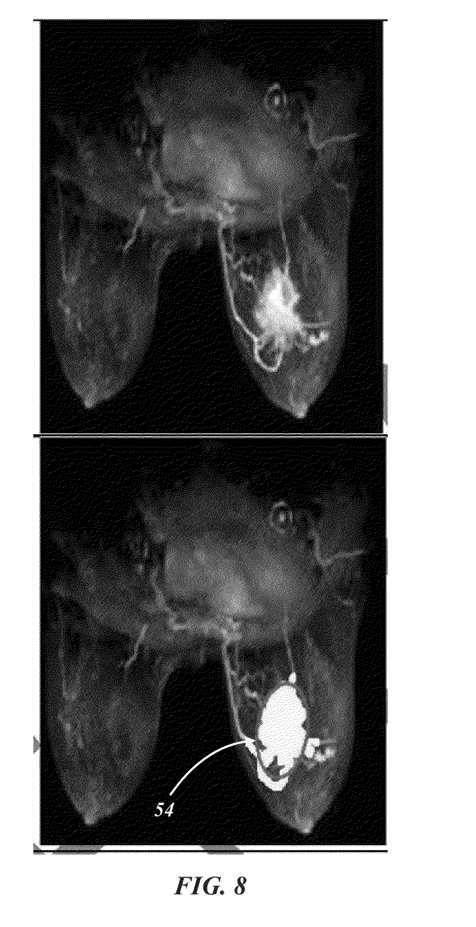

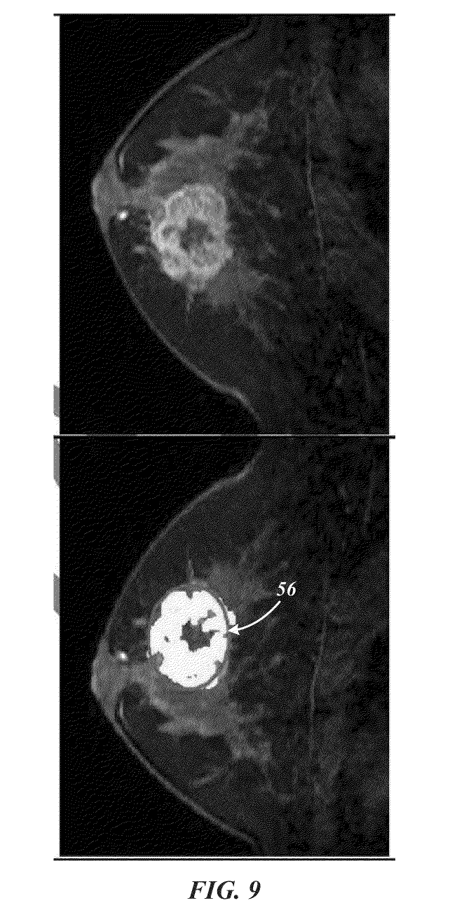

FIG. 2 through FIG. 9 show example results of the approaches described herein for processing an MRI image to detect cancer in a patient. FIG. 2 shows two detected regions 38, 40 in a patient's brain identified as being potential brain cancer tumors. FIG. 3 shows a detected region 42 in a patient's brain identified as being a potential brain cancer tumor. FIG. 4 shows a detected region 44 in a patient's breast identified as being a potential breast cancer. FIG. 5 shows a detected region 46 in a patient's breast identified as being a potential breast cancer. FIG. 6 shows a detected region 48 in a patient's brain identified as being a potential brain cancer. The processing of the MRI image to detect the region 48 took approximately 13 seconds. FIG. 7 shows two detected regions 50, 52 in a patient's brain identified as being potential brain cancer. The processing of the MRI image to detect the regions 50, 52 took approximately 15 seconds. FIG. 8 shows a detected region 54 in a patient's breast identified as being a potential breast cancer. The processing of the MRI image to detect the region 54 took approximately 6 seconds. FIG. 9 shows a detected region 56 in a patient's breast identified as being a potential breast cancer. The processing of the MRI image to detect the region 56 took approximately 5.2 seconds.

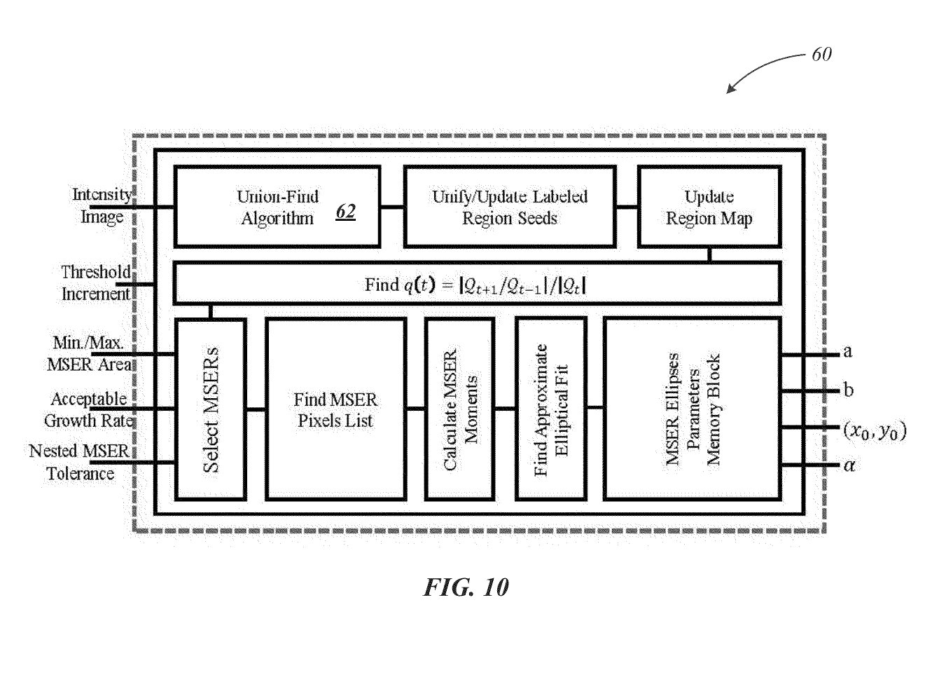

FIG. 10 illustrates a method 60 for MSER detection, in accordance with many embodiments. The method 60 includes the use of an efficient Union-Find algorithm 62 to label each of the extremal regions at each threshold. In the method 60, there are four main parameters that control the detection of the MSERs, namely the maximum and minimum allowable number of pixels of the MSER, the maximum allowable growth rate specified by the stability function, the threshold increment, and the nesting tolerance. Different choices of those parameters yield different detected MSERs. The first two parameters (MinArea and MaxArea) are used to exclude too small or too large MSERs, i.e., all detected MSERs satisfy the condition set forth in equation (2).

MinArea≦Q≦MaxArea Equation (2)

The third parameter, the Maximum Acceptable Growth Rate, specifies how stable the detected MSERs should be, i.e., all detected MSERs must satisfy the condition set forth in equation (3).

q(t)=[Q(t+Δ)/Q(t−Δ)]/Q(t)≦MaxGrowth Equation (3)

The final parameter, the Nesting Tolerance Value, is used to resolve the weaknesses of the MSERs. Since nested MSERs have almost the same center coordinates, any new MSER with its center in the range specified by the tolerance value compared to previously detected and stored MSER will be excluded automatically, i.e., all detected MSERs satisfy the conditions set forth in equation (4) and equation (5).

x₀: [symbol] {(1−0.5τ)xᵢ, (1+0.5τ)xᵢ}. (4)

y₀: [symbol] {(1−0.5τ)yᵢ, (1+0.5τ)yᵢ}. (5)

τ refers to the tolerance value, and xᵢ and yᵢ denotes all previously stored center values of the detected MSERs. This approach, even though relatively simple, has a major drawback, which is the unnecessary computation needed for the calculation of image moments. To predict possible nesting, and hence save all those unnecessary operations, as an alternative approach with far much lower computational cost, for each region, the current growth rate can be compared to the previous growth rate, and if absolute difference is within some range, defined again by the tolerance parameter τ, then this region at the current threshold can be excluded from MSER detection processing. The last parameter, the threshold increment, Δ, can be selected as 5 to speed up the MSER detection process. Approximately, MSER detection with Δ equals to 5 is five times faster than when Δ equals to 1. Finally, since merged regions have the same growth rate from the threshold level they merge and afterwards, only one MSER, corresponding to the region with the seed that comes first in the SeedList is detected and the rest not processed and ignored. This alternative approach saves reduces the number of computations, and hence time and power. The full MSER algorithm implementation consists, therefore, of the following main stages: a) thresholding, b) labeling, c) unifying/updating regions seeds, d) updating region map, e) selection of MSERs, f) MSER pixels, moments, and ellipse parameters, and g) store MSER elliptical fit parameters.

A. Thresholding

The incoming frame (intensity image) is thresholded, starting with threshold of value 255 with Δ increments down to 0, i.e., each frame requires 255/Δ+1 thresholding (e.g. for Δ equals to 5, 52 thresholding processes are required for each frame).

B. Labeling

The Union-Find algorithm 62 is used to label the binary image. The algorithm will output the labeled image, the seed, and the size (the number of pixels with the same label) of each region, plus the number of labels used, respectively referred to as ID, Seeds, SeedsRS, and NumSeeds.

C. Unifying/Updating Region Seeds

This is step is necessary for the system to work properly due to the following rationale. The Union-Find algorithm returns labeled regions and their corresponding sizes and seeds. The seed of each region at this threshold is the first pixel location that the algorithm encounters of every region. Next, due to the threshold increment, previous regions might grow or even merge and new regions might appear. This means that the Union-Find will label those regions with labels, still unique but not necessarily similar to previous labels or with the same seeds. More importantly, since the regions grow/merge, the first pixel location that the Union-Find encounters for the growing region, i.e. its current seed, will be definitely different from the previous seed, even though both refer to the same region. Obviously, for those growing regions, the seed, i.e. first detected pixel of every connected component set, is likely to be different from that at the previous threshold, even though both refer to the same region. To overcome this issue, all seeds that get stored at this threshold, in the Seeds memory, are compared with the seeds previously detected and stored in the SeedList. This is simply done by comparing the labels, stored in ID, at the locations specified by the Seeds at the current threshold, and the stored SeedList. If a match is found, the old seed is maintained, otherwise a new seed is appended to the SeedsList.

D. Updating Region Map

The region map is a dedicated memory that is used to store the seeds' region sizes, consisting of 3×#seeds stored in the SeedList registers, to store the value of [Q(t+Δ)], [Q(t)], and [Q(t−Δ)] for each seed; the values are needed to calculate the stability function for each seed in the SeedList. This is done (for memory reduction and efficiency) instead of recording the region size for every seed in the SeedList at every threshold. With this, if more seeds are appended to the SeedList at threshold t+Δ, then new locations for this new seed are also appended to the RegionMap, where the region size for this threshold is added in the [Q(t+Δ)] while [Q(t)], and [Q(t−Δ)] are filled with ones (to avoid division by zero). Note that since [Q(t+Δ)] is not available at the current threshold t, nor is available for the first threshold, then the calculation of (1) starts at the third threshold, i.e., q(t) is calculated at threshold t+Δ, excluding the first and final threshold values. In this way, the stability function can be easily calculated and this is the reason for the RegionMap memory to have three rows. To elaborate on this, consider the following sample scenario presented in Table 2 below. At the third threshold, in Table 2, q(t) is calculated for the second threshold. At [Q(t)], the two regions defined by Seed#1 and Seed#2 merge, so they have the same size from now on. At the fourth threshold, in Table 2, q(t) is calculated for the third threshold, and note that [Q(t+Δ)] and [Q(t)] at the third threshold are [Q(t)] and [Q(t−Δ)] at the fourth threshold. Because of the detection of a new region, defined by Seed#5, RegionMap list is appended and the size of this new region at [Q(t+Δ)] is filled with its size, while [Q(t)], and [Q(t−Δ)]are filled with ones. At this threshold, regions referred to by Seed#3 and Seed#4 merge so they will have the same region size from now on, etc. Note that at the final threshold, all regions will merge into one with a size M×N.

TABLE 2
Example SeedList and RegionMap Scenario
(a) At the third threshold.

| SeedsList | Seed #1 | Seed #2 | Seed #3 | Seed #4 | blank |
|---|---|---|---|---|---|
| \|Q(t − Δ)\| | 25 | 49 | 102 | 4 | blank |
| \|Q(t)\| | 120 | 120 | 135 | 11 | blank |
| \|Q(t + Δ)\| | 155 | 155 | 173 | 44 | blank |

(b) At the fourth threshold

| | | | | | Seed |
|---|---|---|---|---|---|
| SeedsList | Seed #1 | Seed #2 | Seed #3 | Seed #4 | #5 |
| \|Q(t − Δ)\| | 120 | 120 | 135 | 11 | 1 |
| \|Q(t)\| | 155 | 155 | 173 | 44 | 1 |
| \|Q(t + Δ)\| | 203 | 203 | 244 | 244 | 13 |

E. Selection of MSERs

At this stage, using q(t) previously calculated, in conjunction with [Q(t)] stored in RegionMap, MSERs are selected to satisfy the conditions (2)-(5).

F. MSER Pixels, Moments, and Ellipse Parameters

For every MSER that satisfies the condition in (2)-(5), the Pixels List, i.e., the x and y coordinates for the labeled region, stored in ID, and defined by its seed stored in the SeedList, are used these to calculate the region moments per equation (6).

m_{pq}=Σ_{[x,y] ε R}x^{p}y_{q}, x, y ε R(τ) (6)

x and y denote the pixel coordinates of the region R(τ) at the current threshold. Subsequently, the region can be approximated by the best-fit ellipse. The ellipse equation is given by equation (7).

$$\frac{{( x - {x}_{0} + tan ( α ) ( y - {y}_{0} ) )}^{2}}{{a}^{2} ( 1 + {tan}^{2} ( α ) )} + \frac{{( y - {y}_{0} + tan ( α ) ( x - {x}_{0} ) )}^{2}}{{b}^{2} ( 1 + {tan}^{2} ( α ) )} = 1 , \qquad ( 7 )$$

(x₀, y₀), a, b, and α are the center of gravity (center of the ellipse), the major and minor axis lengths and the angle with respect to the horizontal axis. These ellipse parameters can be calculated from the region moments m00, m01, m10, m11, m02, and m20 as set forth in equation (8) through equation (15).

$$\begin{matrix}{x}_{0} = \frac{{m}_{10}}{{m}_{00}} , & ( 8 ) \\ {y}_{0} = \frac{{m}_{01}}{{m}_{00}} , & ( 9 ) \\ a = \sqrt{2 ( {t}_{1} + {t}_{3} + \sqrt{{t}_{2}^{2} + {( {t}_{3} - {t}_{1} )}^{2}} )} , & ( 10 ) \\ b = \sqrt{2 ( {t}_{1} + {t}_{3} - \sqrt{{t}_{2}^{2} + {( {t}_{3} - {t}_{1} )}^{2}} )} , & ( 11 ) \\ α = 0.5 {tan}^{- 1} ( \frac{{t}_{2}}{{t}_{1} - {t}_{3}} ) , \\ where & ( 12 ) \\ {t}_{1} = \frac{{m}_{20}}{{m}_{00}} - {x}_{0}^{2} , & ( 13 ) \\ {t}_{2} = 2 ( \frac{{m}_{11}}{{m}_{00}} - {x}_{0} {y}_{0} ) , & ( 14 ) \\ {t}_{3} = \frac{{m}_{02}}{{m}_{00}} - {y}_{0}^{2} , & ( 15 )\end{matrix}$$

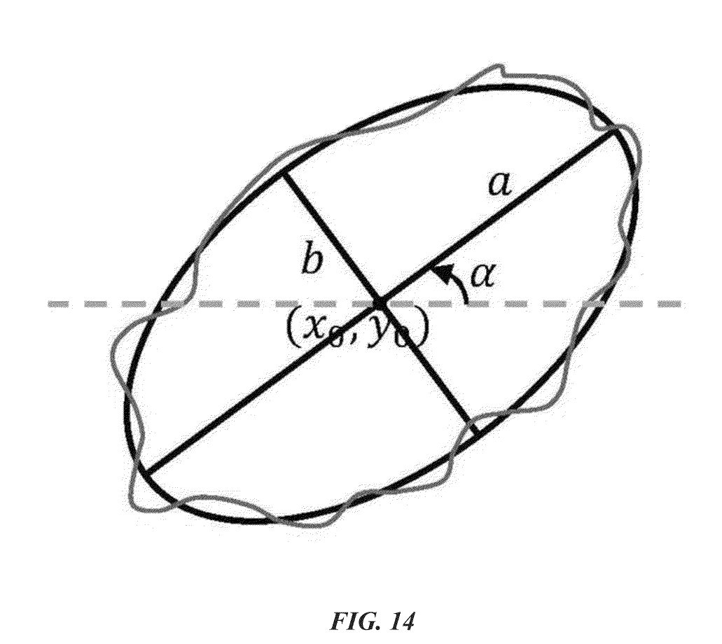

A sample sketch for an irregularly shaped region and its best fit elliptical approximation is shown in FIG. 14. Note that since regions merge will cause the same region sizes to be stored for multiple seeds, which means that if an MSER is detected, multiple detections referring to the same MSER may occur. To avoid this, only the first seed for the merged regions is considered, as discussed above.

G. Store MSER Elliptical Fit Parameters

Finally, instead of storing each MSER Pixels List, which will require a huge memory, parameters of the best-fit ellipses (x₀, y₀, α, a, and b) are stored to be displayed or further monitored. In fact, it's noteworthy that since the elliptical fit parameters are available, they can be used to compute SURF or SIFT descriptors. Depending on the version of the intensity image used, the detector will either detect bright or dark MSERs.

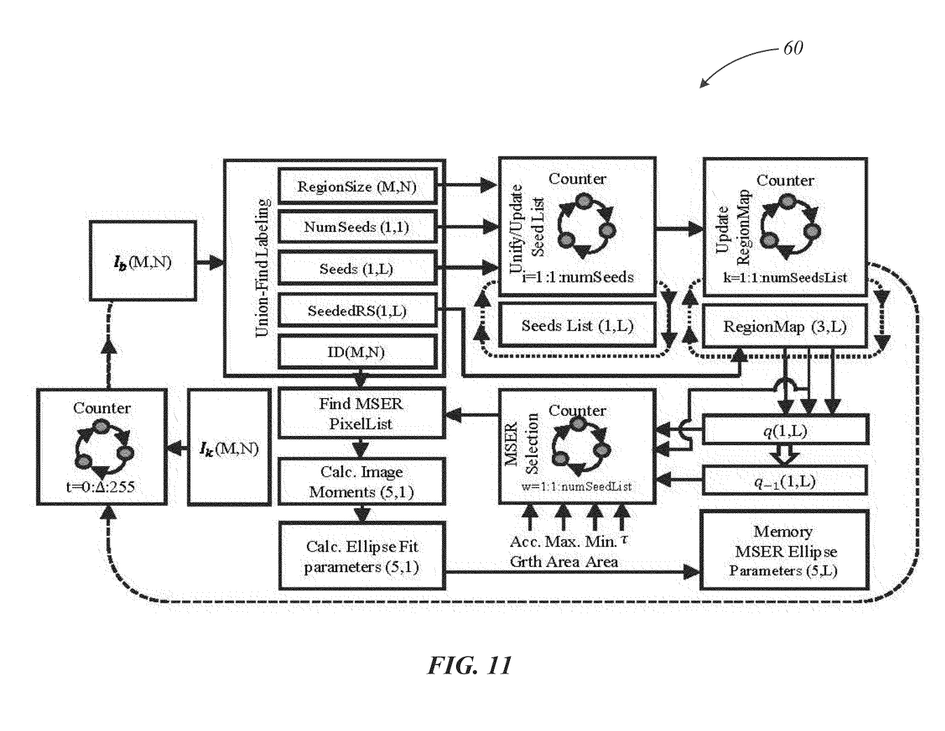

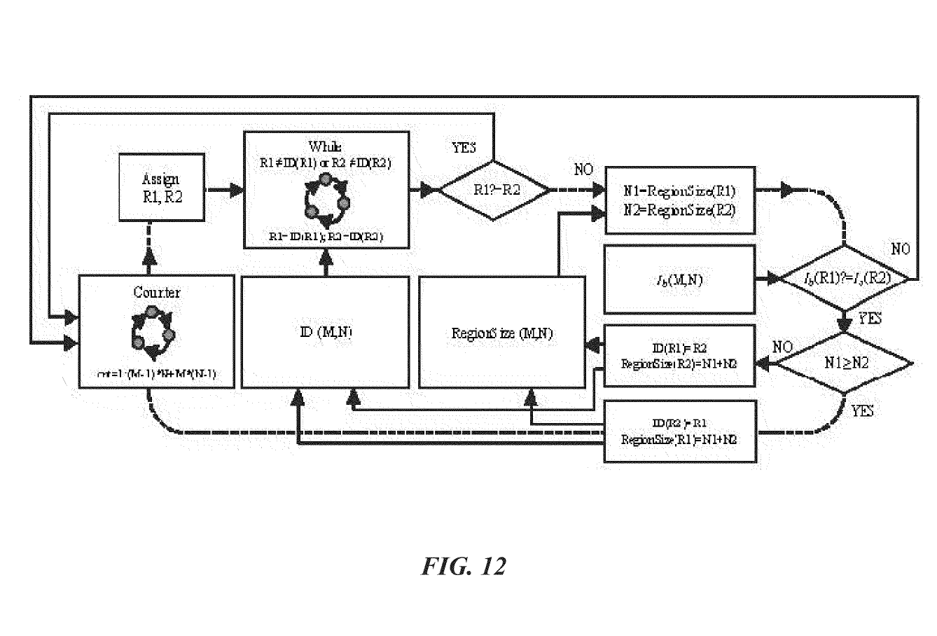

FIG. 11 provides more detailed illustration of the method 60 for MSER detection, in accordance with many embodiments. The MSER detection can be accomplished, as described herein, using 255/Δ+1 thresholding processes to generate resulting binary threshold images. In the following discussion, reference is made to one threshold level, t. The MSER detection method 60 takes the intensity image, of size M×N, and threshold it resulting in an M×N binary threshold image. The binary threshold image is then passed to the Union-Find algorithm to carry out the white spot labeling and will result in a uniquely labeled regions that can be easily identified and hence used for the later MSER processing. The Union-Find Architecture is shown in FIG. 12.

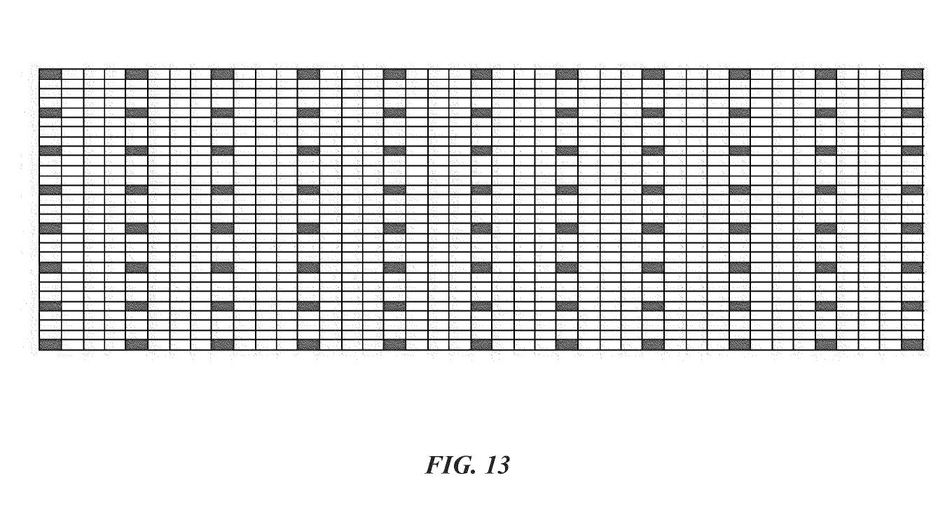

The Union-Find algorithm has two stages of implementation. The first stage uses two M×N memories for the ID and RegionSize matrices. The ID matrix initially labels and assigns every non-zero pixel by an id value, and the RegionSize matrix is filled in these non-zero locations with ones, assuming initially that each pixel is an individual region and not connected to any other pixels. The Union-Find algorithm uses an iterative process. It can be shown that at most M×(N−1)+M×N iterations are sufficient to process all the pixels. Each region's roots are identified and the connected components belonging to every root are assigned the region root as their label. The sizes of the regions, maintained in RegionSize, are incremented to identify the number of pixels that have the same label. At the end of the iterative process, the ID matrix is a uniquely labeled image, and the RegionSize matrix has the size of each region stored at the same locations corresponding to the label of that region's root. In other words, the RegionSize matrix will end up being also labeled by the size of that region. The RegionSize matrix is different from the ID matrix in a major way in which it gets labeled by the sizes of the regions rather than the roots as in the ID matrix. The root is a unique label, while the region size is not, as two different regions might have the same region size, and virtually look connected if they share a boundary in the RegionSize matrix. The usefulness of using the ID and RegionSize matrices becomes evident in the second stage of our Union-Find implementation. Once the first stage is done, one final scan through the ID matrix, our uniquely labeled image, will be done to identify the regions' seeds. In this scanning, the architecture does not pass through all rows and columns, it passes through the one column and skips the next, for example β columns, and continues till it reaches the last column, and similarly for the rows, while storing the set of unique seeds, Seeds, and counting the seeds number. This scanning is illustrated in FIG. 13 for β=3.

The design approach has three main advantages. First, it helps in ignoring small labeled regions that are not that valuable for the MSER detector. Second, once the regions' seeds are identified, their region sizes can be directly located from the RegionSize memory, due to the analogy described earlier. Finally, the scanning speeds up the Union-Find by a factor of more than β², due to locations' skipping, enhancing the speed and the efficiency of the detection of the MSERs. The Union-Find algorithm outputs the labeled image, the detected seeds from scanning, the number of seeds, and their region sizes, defined respectively as ID, Seeds, NumSeeds, and SeedsRS.

At this stage, the current detected seeds, Seeds, are compared with the SeedsList stored from the earlier thresholds, i.e., from thresholds of values t−Δ, t−2Δ, . . . , Δ, 0. This step is used to unify and update the Seeds List, because of the region growing/merging effect, and the detection of new regions. Once the SeedsList gets updated, RegionMap is then filled with the corresponding seed region size from the SeedsRS memory from the Union-Find algorithm. Hence, utilizing [Q(t+Δ)], [Q(t)], and [Q(t−Δ)] stored in the RegionMap memory, the stability function, q(t), for the previous threshold can be calculated in the way described herein. MSERs are then selected by comparing the region size stored in the RegionMap's Q(t) row, with the MinArea and MaxArea MSER control parameters, and in a parallel fashion, q(t) is compared with the Acceptable Growth Rate value, while monitoring the nesting using the tolerance value, τ. The MSERs that get selected satisfy these control parameters. Then, those MSERs are identified by their seeds from the SeedsList, and all pixels coordinates that has the same label as these seeds, that can be identified using the ID memory, are passed to calculate their region's moments and hence their elliptical fit parameters. The elliptical fit parameters are then stored in a dedicated memory, being appended to previously stored elliptical fit parameters from previous thresholds. The entire process is then repeated until the final threshold value is reached.

Systems

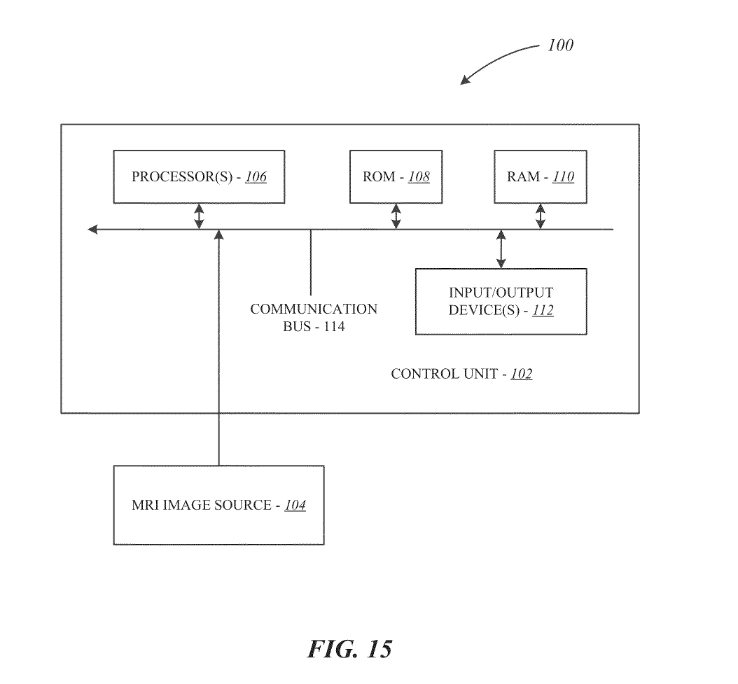

The approaches described herein for processing an MRI image(s) to detect and/or track expansion or contraction of cancer can be implemented on any suitable system. For example, FIG. 15 schematically illustrates a system 100 that can be used to accomplish the approaches described herein. The system 100 includes a control unit 102 and an MRI image source 104.

The control unit 102 includes one or more processors 106, read only memory (ROM) 108, random access memory (RAM) 110, one or more input/output devices 112, and a data bus 114. The ROM 108 can store basic operating system instructions executable by the one or more processors 106 to control basic operations of the control unit 102. The RAM 110 can store instructions executable by the one or more processors 106 to implement the approaches described herein for processing an MRI image(s) to detect and/or track expansion or contraction of cancer. The data bus 114 provides a communication channel interconnecting the one or more processors 106, the ROM 108, the RAM 110, the input/output devices 112, and the MRI image source 104. Any suitable type and number of the input/output device(s) 112 can be used including, but not limited to, a keyboard(s), a display(s), a mouse(s), etc.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A method of processing a magnetic resonance imaging (MRI) image to detect cancer in a patient, the method comprising:
receiving an MRI image of the patient;
forming a series of binary threshold intensity images from the MRI image, each of the series of binary threshold intensity images being based on a respective intensity in a series of intensities;
processing the series of binary threshold intensity images to identify one or more bright extremal regions in which image pixels in the respective binary threshold intensity image have the same value, and for which corresponding image pixels in the MRI image have a higher intensity than surrounding image pixels in the MRI image;
selecting one or more bright maximally stable extremal regions from the identified bright extremal regions based on change in area of one or more respective bright extremal regions for different binary threshold images in the series; and
identifying at least one of the selected one or more bright maximally stable extremal regions as potentially cancerous.

2. The method of claim 1, wherein no bright maximally stable extremal regions having a corresponding image intensities in the MRI image less than a minimum intensity value and/or greater than a maximum intensity value are identified as potentially cancerous.

3. The method of claim 1, wherein no bright maximally stable extremal regions having an area less than a minimum area and/or greater than a maximum area are identified as potentially cancerous.

4. The method of claim 1, wherein no bright extremal regions having a change in area of greater than a maximum area change tolerance for the different images in the series of binary threshold intensity images are selected as the bright maximally stable extremal regions.

5. The method of claim 1, wherein:
no bright maximally stable extremal regions having a corresponding image intensities in the MRI image less than a minimum intensity value and/or greater than a maximum intensity value are identified as potentially cancerous;
no bright maximally stable extremal regions having an area less than a minimum area and/or greater than a maximum area are identified as potentially cancerous; and
no bright extremal regions having a change in area of greater than a maximum area change tolerance for the different images in the series of binary threshold intensity images are selected as the bright maximally stable extremal regions.

6. The method of claim 1, further comprising generating parameters descriptive of the location and size of the at least one potentially cancerous region.

7. The method of claim 6, wherein the parameters define an ellipse approximating the respective potentially cancerous region.

8. The method of claim 1, further comprising:
receiving a second MRI image of the patient;
forming a second series of binary threshold intensity images from the second MRI image, each of the second series of binary threshold intensity images being based on a respective intensity in a second series of intensities;
processing the second series of binary threshold intensity images to identify one or more second image bright extremal regions in which image pixels in the respective binary threshold intensity image have the same value, and for which corresponding image pixels in the second MRI image have a higher intensity than surrounding image pixels in the second MRI image;
selecting one or more second image bright maximally stable extremal regions from the identified second image bright extremal regions based on change in area of one or more respective second image bright extremal regions for different binary threshold images in the second series; and
identifying at least one of the selected one or more second image bright maximally stable extremal regions as corresponding to at least one of the one or more bright maximally stable extremal regions identified as potentially cancerous.

9. The method of claim 8, further comprising determining a change in area of a region identified as potential cancerous based on the MRI image and the second MRI image.

10. The method of claim 1, wherein the one or more bright maximally stable extremal regions identified as potentially cancerous are identified as being potentially one of the group of cancers consisting of: a) bladder cancer, b) breast cancer, c) colon cancer, d) rectal cancer, e) endometrial cancer, f) kidney cancer, g) leukemia, h) lung cancer, i) melanoma cancer, j) non-Hodgkin lymphoma cancer, k) pancreatic cancer, l) prostate cancer, m) thyroid cancer, and n) brain cancer.

11. A system for processing a magnetic resonance imaging (MRI) image to detect cancer in a patient, the system comprising:
one or more processors; and
a tangible memory storage device storing instructions that when executed by the one or more processors cause the system to:
receive an MRI image of the patient;
form a series of binary threshold intensity images from the MRI image, each of the series of binary threshold intensity images being based on a respective intensity in a series of intensities;
process the series of binary threshold intensity images to identify one or more bright extremal regions in which image pixels in the respective binary threshold intensity image have the same value, and for which corresponding image pixels in the MRI image have a higher intensity than surrounding image pixels in the MRI image;
select one or more bright maximally stable extremal regions from the identified bright extremal regions based on change in area of one or more respective bright extremal regions for different binary threshold images in the series; and
identify at least one of the selected one or more bright maximally stable extremal regions as potentially cancerous.

12. The system of claim 11, wherein no bright maximally stable extremal regions having a corresponding image intensities in the MRI image less than a minimum intensity value and/or greater than a maximum intensity value are identified as potentially cancerous.

13. The system of claim 11, wherein no bright maximally stable extremal regions having an area less than a minimum area and/or greater than a maximum area are identified as potentially cancerous.

14. The system of claim 11, wherein no bright extremal regions having a change in area of greater than a maximum area change tolerance for the different images in the series of binary threshold intensity images are selected as the bright maximally stable extremal regions.

15. The system of claim 12, wherein:
no bright maximally stable extremal regions having a corresponding image intensities in the MRI image less than a minimum intensity value and/or greater than a maximum intensity value are identified as potentially cancerous;
no bright maximally stable extremal regions having an area less than a minimum area and/or greater than a maximum area are identified as potentially cancerous; and
no bright extremal regions having a change in area of greater than a maximum area change tolerance for the different images in the series of binary threshold intensity images are selected as the bright maximally stable extremal regions.

16. The system of claim 11, wherein the instructions, when executed by the one or more processors, further cause the system to generate parameters descriptive of the location and size of the at least one potentially cancerous region.

17. The system of claim 16, wherein the parameters define an ellipse approximating the respective potentially cancerous region.

18. The system of claim 11, wherein the instructions, when executed by the one or more processors, further cause the system to:
receive a second MRI image of the patient;
form a second series of binary threshold intensity images from the second MRI image, each of the second series of binary threshold intensity images being based on a respective intensity in a second series of intensities;
process the second series of binary threshold intensity images to identify one or more second image bright extremal regions in which image pixels in the respective binary threshold intensity image have the same value, and for which corresponding image pixels in the second MRI image have a higher intensity than surrounding image pixels in the second MRI image;
select one or more second image bright maximally stable extremal regions from the identified second image bright extremal regions based on change in area of one or more respective second image bright extremal regions for different binary threshold images in the second series; and
identify at least one of the selected one or more second image bright maximally stable extremal regions as corresponding to at least one of the one or more bright maximally stable extremal regions identified as potentially cancerous.

19. The system of claim 18, wherein the instructions, when executed by the one or more processors, further cause the system to determine a change in area of a region identified as potential cancerous based on the MRI image and the second MRI image.

20. The system of claim 11, wherein the one or more bright maximally stable extremal regions identified as potentially cancerous are identified as being potentially one of the group of cancers consisting of: a) bladder cancer, b) breast cancer, c) colon cancer, d) rectal cancer, e) endometrial cancer, f) kidney cancer, g) leukemia, h) lung cancer, i) melanoma cancer, j) non-Hodgkin lymphoma cancer, k) pancreatic cancer, l) prostate cancer, m) thyroid cancer, and n) brain cancer.
